(19) United States
(12) Patent Application Publication
(10) Pub. No.: US 2006/0000476 A1
(43) Pub. Date: Jan. 5, 2006

(54) Retaining system for securing a ventilation mask

(76) Inventor: M. Ramez Salem, Chicago, IL (US)

Correspondence Address: BANNER & WITCOFF, LTD., 1001 G STREET, N.W., WASHINGTON, DC, 20001-4597, US

(21) Appl. No.: 11/174,429

(22) Filed: Jul. 1, 2005

Related U.S. Application Data
(60) Provisional application No. 60/585,422, filed on Jul. 1, 2004.

(51) Int. Cl. A62B 18/02; A62B 7/00; A61M 15/08

(52) U.S. Cl. 128/206.21; 128/207.11; 128/206.24

(57) ABSTRACT

A ventilation mask system including a mask, such as a mask for use with intermittent positive pressure ventilation, and an associated retainer that allows the mask to be securely and comfortably held against a patient's face. The mask is provided with a retaining strip that includes one mating portion of a mating hook-and-loop fastener system. A retainer corresponding to the mask includes the other mating portion of the hook-and-loop fastener system. Accordingly, when the hook-and-loop material on the retainer is mated with the corresponding hook-and-loop material provided on the ventilation mask, the mask will be securely held in place on the patient's face.

### RELATED APPLICATIONS

This application is a continuation of U.S. Provisional Patent Application No. 60/585,422 entitled “Ventilation System For Intermittent Positive Pressure Ventilation Masks,” filed on Jul. 1, 2004, and naming M. Ramez Salem as inventor, which application is incorporated entirely herein by reference.

### FIELD OF THE INVENTION

The present invention relates to ventilation masks and in particular to retainers for securing a ventilation mask to a user. In preferred embodiments, the present invention is directed to ventilation systems that comprise a mask to be used for intermittent positive pressure ventilation or spontaneous breathing ventilation, and a retainer.

### BACKGROUND OF THE INVENTION

Ventilation masks, such those used with intermittent positive pressure ventilation (IPPV) and spontaneous breathing ventilation, are employed for a variety of medical applications. For example, when a patient receives anesthetic gas during a medical operation, the gas typically will be delivered through a mask using intermittent positive pressure ventilation. Unlike other types of masks, such as continuous positively airway pressure (CPAP) masks, masks used for intermittent positive pressure ventilation are oronasal masks that cover both a patient's face and nose, in order to completely access the patient's airways. The anesthetic gas then is delivered through the mask with either intermittent positive pressure or spontaneous breathing. These ventilation masks thus are used with intermittent positive pressure ventilation masks and spontaneous breathing ventilation to provide patients with anesthetic gases and oxygen.

FIGS. 1A and 1B illustrate a conventional intermittent positive pressure ventilation mask 101. As seen in these figures, the mask 101 includes a dome 103, a balloon cushion 105, a cushion valve 107, and a hose connector 109. The dome 103 is open at the bottom so as to form a cup, and typically is made from a hard plastic or similar material. As seen in FIG. 1B, the dome 103 may have a pear or oval shape, so that it can comfortably cover both a patient's nose and mouth at the same time. The balloon cushion 105 comprises a bag or roll of soft, flexible material that extends around the periphery of the dome 103. Air can be introduced into the inside of the balloon cushion 105 through the cushion valve 107.

The hose connector 109 connects to a hose through which the desired gas is delivered into the mask 101, and forms an aperture into the inside of the dome 103. The hose connector 109 is sized to securely fit the hose delivering the desired gas into the mask 101. As shown in FIGS. 1A and 1B, the dome 103 includes a flattened extremity that forms a shelf 111 around the hose connector 109. The shelf 111 facilitates the use of a conventional retaining system for holding the mask 101 in place on a patient's face. As shown in FIGS. 2A and 2B, a post ring 201 (sometimes also referred to as a “hook ring” or “collar ring”) is employed by this conventional retaining system. The post ring 201 includes a base 203 and four posts 205 extending from the base 203. As seen in these figures, the post ring 201 is positioned such that the base of the post ring 201 rests on the shelf 111 of the dome 103. In this position, the posts 205 extend from the post ring 201 away from the dome 103. This conventional retaining system also employs a retainer, such as the retainer 301 shown in FIG. 3 (FIG. 3 is not drawn to scale). The head band 301 includes a central portion 302 and two pairs of opposing extensions 303. Each extension 303 includes a plurality of perforations 305 that are generally evenly spaced apart and that are disposed along at least a portion of the length of the extension 303.

To secure the mask 101 to a patient's face, the central portion 302 of the head band 301 is positioned behind the patient's head. After the mask 101 has been placed on the patient's face at the desired position, each of the extensions 303 is drawn around the patient's head to extend over the post ring 201. More particularly, as shown in FIG. 4, each extension 303 is positioned over a post 205 so that the post 205 passes through a perforation 305 in the extension 303. The extensions 303 generally are formed from an elastic material, and the perforations 305 are sized to receive the posts 205 in a manner that allows each post 205 to be elastically retained with respect to the respective engaging extension 303. By selecting the perforation 305 that is to receive the post 205 corresponding to the extension 303, a medical care provider can control the tension of the extension 303. In this manner, the mask 101 may be securely retained on the patient's face.

While this conventional retaining system is commonly employed, it suffers a number of drawbacks and disadvantages. Unlike CPAP masks, which typically are used in a home environment and manipulated by the patient, masks used for intermittent positive pressure ventilation and spontaneous breathing ventilation typically are employed in a hospital or other professional medical environment. Accordingly, the medical service provider using these masks should secure the mask to the face of a patient while wearing surgical gloves. The conventional retainer 301 was developed before the use of surgical gloves by anesthetists was common, and thus is difficult to use while wearing surgical gloves.

There are still other disadvantages to conventional mask retaining systems; for example, the post ring 201 is a separate component from the mask 101, and must be manufactured separately, thus increasing manufacturing costs. Additionally, the post ring 201 easily can become lost or disassociated from the mask 101.

Still further, depending upon the spacing between the perforations 305 formed in the extensions 303, the medical care provider may be forced to choose between positioning the extensions 303 with too much tension and positioning the extensions 303 with too little tension. If the extensions 303 are fastened to the posts 205 with too much tension, the mask 101 will be forced down against the patient's face, creating discomfort for the patient. Alternately, if the extensions 303 are fastened to the posts 205 with too little tension, the mask 101 may not be securely pressed against the patient's face, and thus may come loose.

### BRIEF SUMMARY OF THE INVENTION

Various embodiments of the invention provide a mask, preferably for use with intermittent positive pressure ventilation or spontaneous breathing ventilation, and an associated retainer that allows the mask to be securely and comfortably held against a patient's face. The mask and retainer together form a ventilation system. In accordance with the invention, a mask is provided with a retaining strip that comprises one mating portion of a mating hook-and-loop fastener system. A retainer corresponding to the mask includes the other mating portion of the hook-and-loop fastener system. Accordingly, when the hook-and-loop material on the retainer is mated with the corresponding hook-and-loop material provided on the mask, the mask will be securely held in place on the patient's face.

### BRIEF DESCRIPTION OF THE DRAWINGS

FIG. 1A is a side elevation of a conventional mask for use with intermittent positive pressure ventilation.

FIG. 1B is a plan view of the conventional mask for use with intermittent positive pressure ventilation shown in FIG. 1A.

FIG. 2A is a perspective view of the mask shown in FIGS. 1A and 1B, including a post ring disposed in a normal operating position with respect to the mask.

FIG. 2B is a plan view of the mask and post ring as illustrated in FIG. 2A.

FIG. 3 is a perspective view (not to the same scale as FIGS. 1A, 1B, 2A, and 2B) of a retainer used with a conventional mask.

FIG. 4 is a plan view of a conventional ventilation system that includes the mask illustrated in FIGS. 1A and 1B, shown with the retainer illustrated in FIG. 3 in an operating position.

FIG. 5A is a side elevation of mask for use with intermittent positive pressure ventilation in accordance with the invention.

FIG. 5B illustrates a plan view of the mask shown in FIG. 5A.

FIG. 6 (not to the same scale as FIGS. 5A and 5B) is a plan view of a retainer useful in connection with the mask shown in FIG. 5.

FIG. 7 is a partial front elevation, enlarged with respect to FIG. 6, of the retainer shown in FIG. 6.

FIG. 8 is a plan view of the ventilation system of the invention, including the mask shown in FIGS. 5A and 5B and the retainer of FIG. 6, the retainer being shown in an operating position.

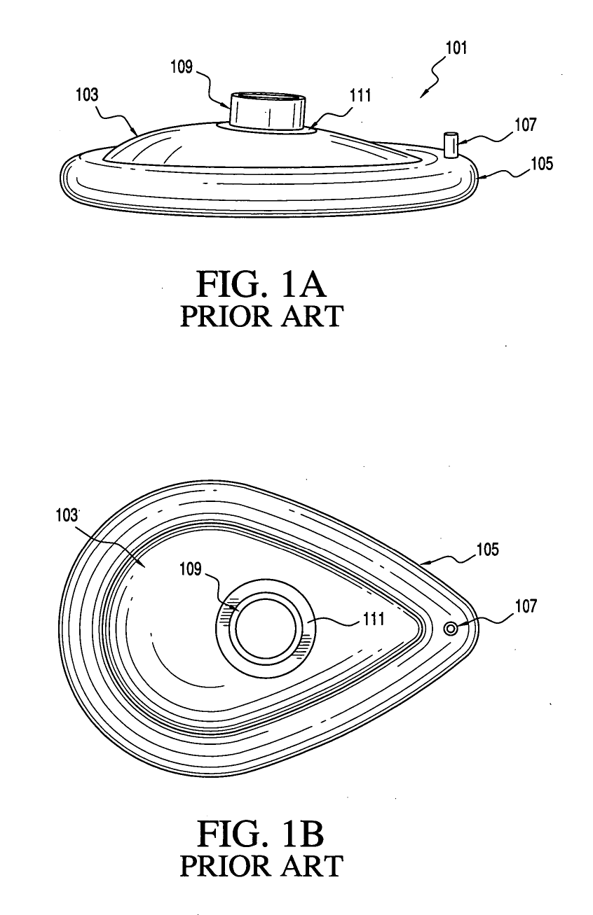

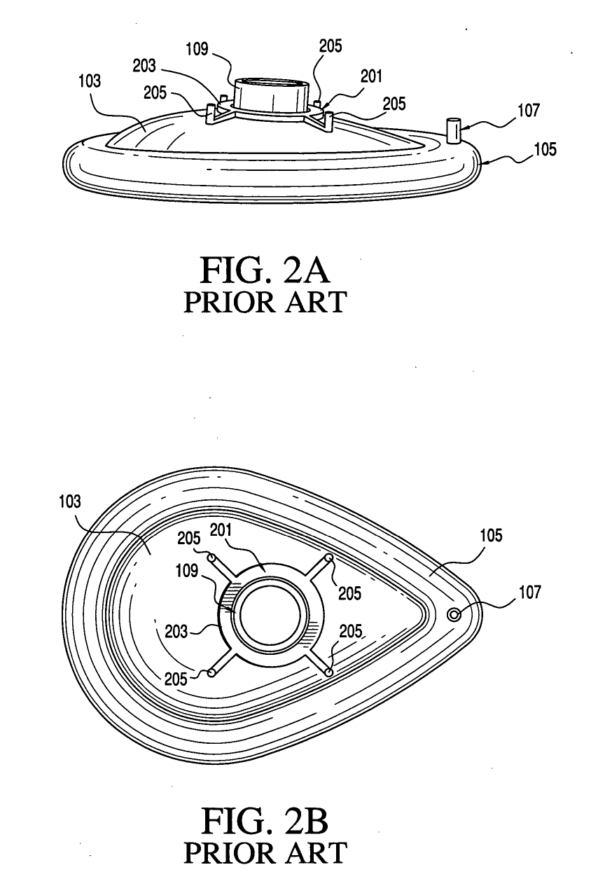

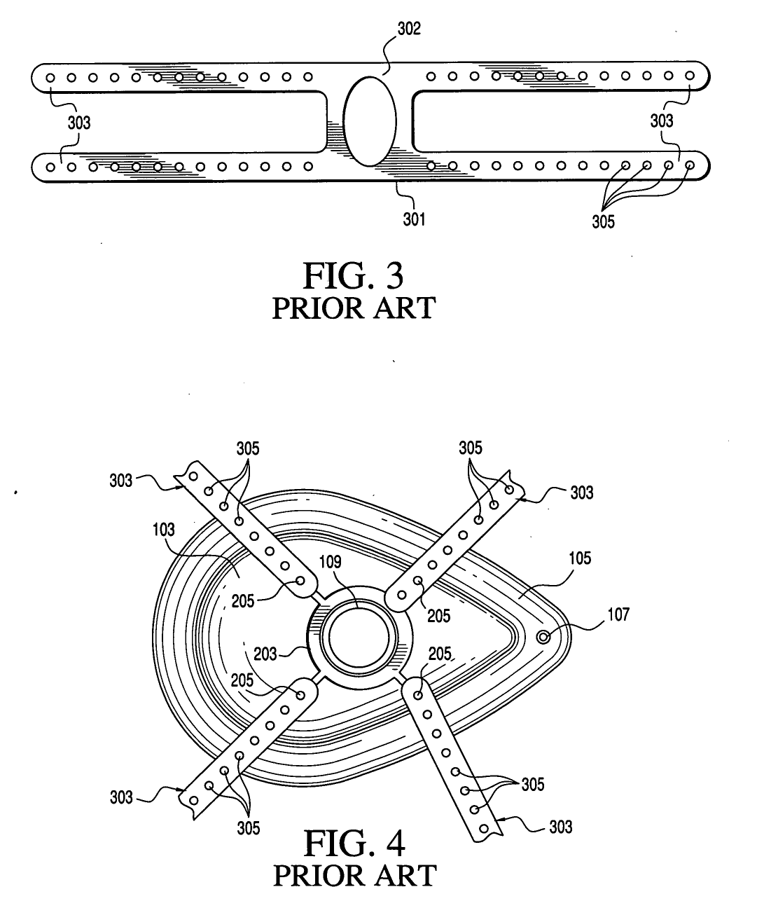

It should be understood that references herein to the orientation of the mask and retainer in the various views are for the purposes of providing relative descriptions; in practice, the mask and retainer may be employed omnidirectionally.

### DETAILED DESCRIPTION OF THE PREFERRED EMBODIMENTS

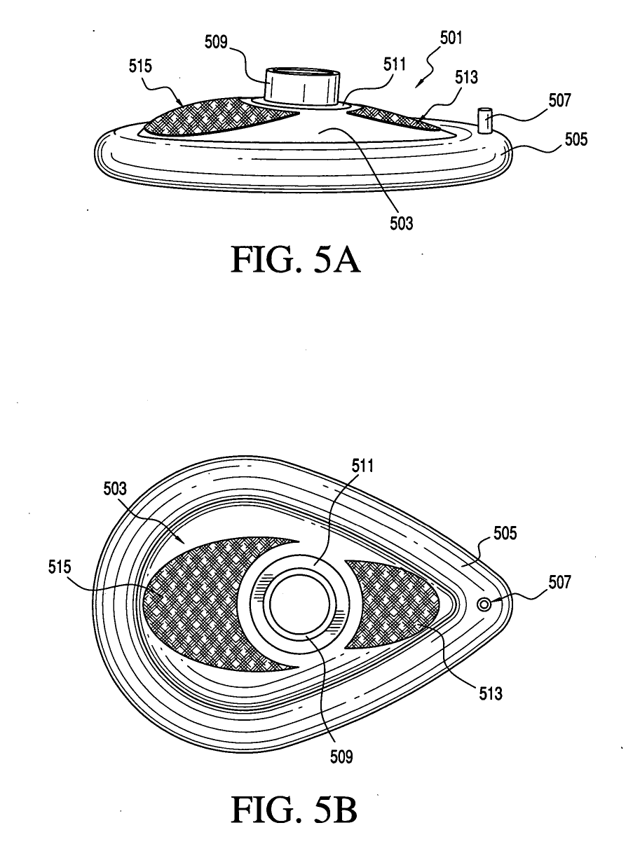

As seen in FIGS. 5A and 5B, the mask 501 includes a dome 503, a balloon cushion 505, a cushion valve 507 and a hose connector 509. The dome 503 is open at the bottom so as to form a cup. The dome 503 may be made from any desirable material, such as, for example, a hard plastic, rubber, or similar material. The dome 503 should be formed with a material that will not react (or will not strongly react) in normal use with the desired gas intended to be provided through the mask 501. As seen in FIG. 5B, the dome 503 may have a pear or oval shape, so that the dome 503 can be comfortably placed over both a patient's nose and mouth at the same time. It should be appreciated, however, in other embodiments of the invention the dome 503 may have a different shape.

The balloon cushion 505 may be formed from any suitable material. The balloon cushion 505 extends around the periphery of the dome 503. In the illustrated embodiment, the balloon cushion 505 is formed of a bag or roll of a soft, flexible material, such as a soft plastic. In this embodiment, air (or another suitable gas) can be introduced into the balloon cushion 505 through the cushion valve 507, to inflate the balloon cushion 505. The cushion 505 alternatively may be composed of any suitable alternative cushioning material, such as a solid gel, a solid piece of soft material (such as rubber), or a combination of different structures.

The hose connector 509 connects to a hose (not shown) through which the desired gas is delivered into the mask 501. The hose will be connected to the breathing circuit of an anesthetic system or a resuscitation bag. These types of anesthetic systems and resuscitation bags are well known in the art, and thus will not be discussed here in further detail.

Returning now to FIG. 5, the hose connector 509 forms an aperture into the inside of the dome 503. The hose connector 509 is sized to be securely fitted to the hose that delivers the desired gas into the mask 501, generally via the use of a suitable fitting. In the illustrated embodiment, the dome 503 includes a shelf 511 surrounding the hose connector 509. While the shelf 511 is not necessary in the context of the invention, by employing this configuration the mask may be manufactured using the same molds and tooling as are used to manufacture conventional masks employed for intermittent positive pressure ventilation. The shelf 511 alternatively may be omitted from the dome 503.

As also seen in FIGS. 5A and 5B, the mask 501 is provided with retaining strips 513, 515, each of which comprises a first mating portion of a pair of mating portions of a hook-and-loop fastener system. The hook-and-loop material for this type of fastener system may be formed from an array of closely spaced hook structures or, alternately, from an array of closely spaced structures forming loops. When a layer of hook-and-loop fastener material formed from closely spaced hooks is pressed against a layer of hook-and-loop fastener material formed from closely spaced loops, the hooks will hook around the loops, thereby removably fastening the two layers together. Hook-and-loop fastener materials are commercially available, for example, under the trademark VELCRO®. The retaining strips are preferably fastened to the mask using adhesive.

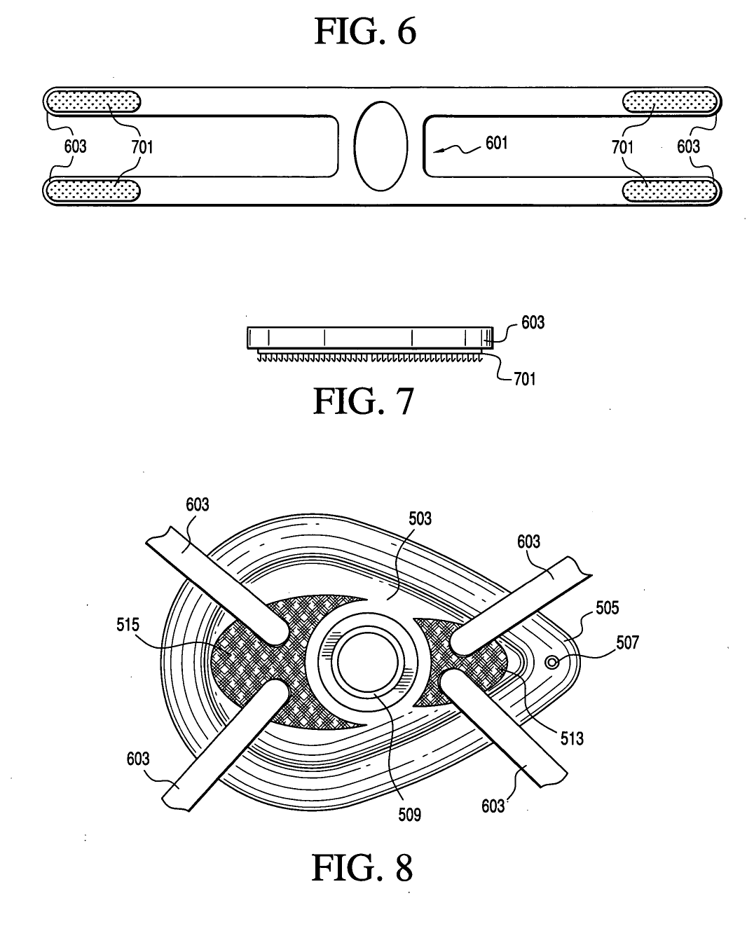

As seen in FIG. 6, the retainer 601 includes a plurality of extensions 603. In the illustrated embodiment, the retainer 601 includes two pairs of opposing extensions 603. The retainer 601 may be formed of an elastic material, such as rubber, leather, or other suitable material. As shown in FIGS. 6 and 7, each extension 603 is provided with a retaining strip 701 that comprises a second mating portion of a pair of hook-and-loop fasteners, the second mating portion being mateably and releasably engagable with the first mating portions of the strips 513, 515 on the mask 501. The strips 701 preferably are secured to the retainer with adhesive.

In operation, such as during a medical operation (e.g., for the induction of an anesthetic gas before a surgical procedure or for the emergence of the patent from an anesthetic after a surgical procedure), the center portion of the retainer 601 (not shown in FIG. 8) is placed behind the patient's head. After the mask 501 is placed at a desired position on the patient's face, the medical care provider draws each of the extensions 603 around the patient's face and over the mask 501, so that each retaining strip 701 of hook-and-loop fastener material engages one of the retaining strips 513 and 515 of the mask 501, as shown in FIG. 8. In this manner, the mask 501 will be securely held to the patient's face. The medical care provider can then perform a medical procedure on the patient after the patient has received the gases as desired (or the patient can emerge from an anesthetic after the medical procedure has been completed).

The mask and retainer thus form a ventilation system, by which is contemplated a system that allows any desired gas to be supplied to a patient. This system offers a number of advantages over the prior art. For instance, the mask readily may be secured using one hand, and can easily be secured by a person wearing protective gloves. The position of each extension 603 relative to the mask 501 may be changed in very small increments, thereby allowing the medical care provider to optimize security and comfort. Moreover, by extending the retaining strips 701 over a substantial portion of the length of the extensions 603, a single size retainer 601 can be used with patients with a variety of different head sizes. After use, the mask 501 can be discarded or sterilized for future use.

While particular embodiments of the invention have been described in detail above, it should be appreciated that various other embodiments of the invention may have alternate or additional features. For example, in the illustrated embodiments, each extension 603 has only a single retaining strip 701 of a hook-and-loop fastener material provided on one side of the extension 603. In alternate embodiments of the invention, however, a retaining strip 701 of a hook-and-loop fastener material may be provided on each side of one or more of the extensions 603. By providing a retaining strip 701 of hook-and-loop fastener material on both sides of the extensions 603, a medical provider will not need to adjust the orientation of the extensions 603 prior to use.

Also, while FIGS. 5A, 5B and 8 show the use of two retaining strips 513 and 515 of a hook-and-loop fastener material disposed on the mask, still other embodiments of the invention may employ a single strip of hook-and-loop fastener material arranged over the mask 501 or conversely three or more strips of hook-and-loop fastener material arranged over the mask 501.

Also, while particular examples of the invention have been described with respect to a mask used for intermittent positive pressure ventilation, it should be appreciated that various embodiments of the invention may be employed to securely retain other types of oronasal ventilation masks that fit over the patient's nose and mouth. Further embodiments of the invention may include alternate or additional modifications and features. Accordingly, the invention should not be deemed limited in scope to the preferred embodiments described herein, and all suitable modifications and equivalents should be considered as falling with the scope of the appended claims.

What is claimed is:

1. A ventilation mask system, comprising:
an oronasal ventilation mask;
a retaining strip provided on the ventilation mask, the retaining strip having a first mating portion of a pair of mating portions of a hook-and-loop fastener system; and
a retainer having a second portion of the pair of mating portions of the hook-and-loop fastener system.

2. The ventilation mask system recited in claim 1, further comprising:
a second retaining strip provided on the ventilation mask, the second retaining strip having the first mating portion of the pair of mating portions of the hook-and-loop fastener system.

3. The ventilation mask system recited in claim 2, wherein the retainer includes
a center portion;
a first extension having the second mating portion of the pair of mating portions of the hook-and-loop fastener system provided on one side thereof; and
a second extension having the second mating portion of the pair of mating portions of the hook-and-loop fastener system provided on one side thereof.

4. The ventilation mask system recited in claim 2, wherein the retainer includes
a first extension having the second mating portion of the pair of mating portions of the hook-and-loop fastener system provided on opposites sides thereof; and
a second extension having the second mating portion of the pair of mating portions of the hook-and-loop fastener system provided on opposite sides thereof.

5. The ventilation mask system recited in claim 2, wherein
the ventilation mask includes a dome defining an aperture therein,
the first retaining strip is provided on the dome at a first side of the aperture, and
the second retaining strip is provided on the dome at a second side of the aperture opposite the first side of the aperture.

6. The ventilation mask system recited in claim 2, further comprising
a third retaining strip provided on the ventilation mask, the third retaining strip having the first mating portion of the pair of mating portions of the hook-and-loop fastener system.

7. The ventilation mask system recited in claim 1, wherein
the ventilation mask includes a dome defining an aperture therein,
the retaining strip is provided on the dome encircling the aperture.

8. The ventilation mask system recited in claim 1, wherein the retainer is formed of an elastic material.

9. The ventilation mask system recited in claim 1, further comprising an anesthetic system or a resuscitation bag.

10. A ventilation mask, comprising:
an oronasal dome defining an aperture therein; and
a retaining strip having a first mating portion of a pair of mating portions of a hook-and-loop fastener system.

11. The ventilation mask recited in claim 10, further comprising:
a second retaining strip having the first mating portion of the pair of mating portions of the hook-and-loop fastener system.

12. The ventilation mask recited in claim 11, wherein
the first retaining strip is provided on the dome at a first side of the aperture, and
the second retaining strip is provided on the dome at a second side of the aperture opposite the first side of the aperture.

13. The ventilation mask recited in claim 10, wherein
the ventilation mask includes a dome defining an aperture therein,
the retaining strip is provided on the dome encircling the aperture.

14. A method of securing a ventilation mask to a patient, comprising:
positioning a retainer proximal to a patient, such that
the retainer includes a center portion, at least one first extension on a first side of the center, and at least one second extension on a second side of the center portion opposite the first side, the first and second extensions having a second mating portion of a pair of mating portions of a hook-and-loop fastener system;
positioning a ventilation mask on the patient, such that the ventilation mask includes at least one retaining strip having a first mating portion of a pair of mating portions of a hook-and-loop fastener system;
drawing the first and second extensions around a head of the patient and over the ventilation mask; and
fastening the first mating portion of the at least one retaining strip to the second portions of the first and second extensions.

15. The method of securing a ventilation mask recited in claim 14,
wherein
the ventilation mask includes a first retaining strip and a second retaining strip,
the retainer has an upper first extension, an upper second extension, a lower first extension and a lower second extension; and
further comprising
fastening the first mating portion the first retaining strip to the second mating portions of the upper first and second extensions, and
fastening the first mating portion the second retaining strip to the second mating portions of the lower first and second extensions.

16. The method of securing a ventilation mask recited in claim 14, further comprising incrementally changing a position at which at least one of the extensions is fastened to the at least one retaining strip.

17. The method of securing a ventilation mask recited in claim 14, further comprising positioning the retainer proximal to a patient, positioning the ventilation mask on the patient, drawing the first and second extensions around the patient's head and over the ventilation mask, and fastening the first mating portion of the at least one retaining strip to the second portions of the first and second extensions using one hand.

18. A method of performing a medical operation, comprising:
securing a mask to a patient by
positioning a retainer proximal to a patient, such that the retainer includes a center portion, at least one first extension on a first side of the center, and at least one second extension on a second side of the center portion opposite the first side, the first and second extensions having a second mating portion of a pair of mating portions of a hook-and-loop fastener system,
positioning the mask on the patient, such that the mask includes at least one retaining strip having a first mating portion of a pair of mating portions of a hook-and-loop fastener system,
drawing the first and second extensions around a head of the patient and over the mask, and
fastening the first mating portion of the at least one retaining strip to the second portions of the first and second extensions;
providing one or more gases to the patient through the mask; and
performing a medical procedure.

19. The method of performing a medical operation recited in claim 18, further comprising disposing of the mask after the medical procedure is completed.

20. The method of performing a medical operation recited in claim 18, further comprising providing the one or more gases to the mask using intermittent positive pressure ventilation.

21. The method of performing a medical operation recited in claim 18,
wherein
the mask includes a first retaining strip and a second retaining strip,
the retainer has an upper first extension, an upper second extension, a lower first extension and a lower second extension; and
further comprising
fastening the first mating portion the first retaining strip to the second mating portions of the upper first and second extensions, and
fastening the first mating portion the second retaining strip to the second mating portions of the lower first and second extensions.

22. The method performing a medical operation recited in claim 18, further comprising incrementally changing a position at which at least one of the extensions is fastened to the at least one retaining strip.

23. The method of performing a medical operation recited in claim 18, further comprising positioning the retainer proximal to the patient, positioning the mask on the patient, drawing the first and second extensions around a head of the patient and over the mask, and fastening the first mating portion of the at least one retaining strip to the second portions of the first and second extensions using one hand.

24. The method of performing a medical operation recited in claim 18, further comprising providing the one or more gases to the mask using spontaneous breathing ventilation.
